(19) United States
(12) Patent Application Publication
(10) Pub. No.: US 2026/0130760 A1
(43) Pub. Date: May 14, 2026

(54) Device and Procedure for Mitral Valve Clip Removal and Subsequent Delivery of a Transcatheter Mitral Valve Implantation

(71) Applicant: Evalve, Inc., Santa Clara, CA (US)

(72) Inventors: Shengmin Mei, Fremont, CA (US); Preston Huddleston, St. Paul, MN (US); Laura M. Kalvass, Mountain View, CA (US); Lauren Troxler Harvey, Austin, TX (US); Lua T. Nguyen, Sunnyvale, CA (US); Tracee Elizabeth Johnson Eidenschink, Wayzata, MN (US); Theodore Paul Dale, Corcoran, MN (US); Kevin P. Griffin, Elk River, MN (US); Michael J. Urick, Chaska, MN (US); Roisin H. Verbael, Redwood City, CA (US); Richard Thomas Childs, San Francisco, CA (US)

(73) Assignee: Evalve, Inc., Santa Clara, CA (US)

(21) Appl. No.: 19/442,510

(22) Filed: Jan. 7, 2026

Related U.S. Application Data
(63) Continuation of application No. 17/475,692, filed on Sep. 15, 2021.
(60) Provisional application No. 63/079,367, filed on Sep. 16, 2020.

(51) Int. Cl. A61F 2/24

(52) U.S. Cl. CPC A61F 2/2427; A61F 2/243; A61F 2220/0016

(57) ABSTRACT

Devices, systems and methods for removal of a mitral valve clip and subsequent delivery of a mitral valve implantation in a mitral valve replacement procedure. A modular port accessory device having a distal connector configured to be compatible with corresponding connectors on an existing therapeutic device, wherein the port accessory can be selectively attached to a proximal end portion connector of a proximal end portion of a catheter assembly of an existing therapeutic device. The catheter assembly is introduced to a target site within the patient's anatomy, and remains in place throughout a multistep therapeutic procedure for use with other therapeutic devices, all while allowing hemostasis to be controlled during use ad interchange of multiple therapeutic devices.

### CROSS-REFERENCE TO RELATED APPLICATIONS

This application is a continuation of U.S. application Ser. No. 17/475,692, filed on Sep. 15, 2021, which claims the benefit of and priority to U.S. Provisional Patent Application No. 63/079,367 , filed Sep. 16, 2020, the disclosures of which are incorporated herein by reference.

### FIELD OF THE INVENTION

Embodiments of the present invention generally relate to medical devices and methods. More particularly, the present invention relates to devices, systems, kits, and methods for facilitating multi-step procedures for removing from and subsequently delivering implanted prostheses to heart valves without the need for establishing access to the target site multiple times.

### BACKGROUND

Mitral regurgitation (MR) is a commonly encountered valvular disorder and prevalence increases with age. In this condition, blood regurgitates abnormally from the left ventricle into the left atrium during cardiac systole, and this condition can result in numerous adverse consequences such as heart failure due to left ventricular dysfunction, atrial fibrillation, pulmonary hypertension, and death. Published guidelines recommend surgical or transcatheter correction of mitral regurgitation to improve the clinical condition.

The most common treatments for mitral valve regurgitation rely on valve replacement or repair including leaflet and annulus remodeling, the latter generally referred to as valve annuloplasty. One technique for mitral valve repair which relies on suturing adjacent segments of the opposed valve leaflets together is referred to as the “bowtie” or “edge-to-edge” technique. While all these techniques can be effective, they usually rely on open heart surgery where the patient's chest is opened, typically via a sternotomy, and the patient is placed on cardiopulmonary bypass. The need to both open the chest and place the patient on bypass is traumatic and has associated high mortality and morbidity.

In some patients, a fixation device can be installed into the heart using minimally invasive techniques. The fixation device can hold the adjacent segments of the opposed valve leaflets together and may reduce mitral valve regurgitation. Transcatheter correction of mitral regurgitation by implantation of a mitral valve clip (in particular the MitraClip® system from Abbott Vascular, Santa Clara, California, USA) is one example of a fixation device that has become a standard therapy for patients at high risk for open surgical corrective procedures. Such clip implantation procedures are performed through a guiding catheter that is inserted into the right femoral vein. One or more mitral valve clips can be delivered through the guiding catheter and implanted to re-approximate the anterior and posterior mitral leaflets (often referred to as an “edge-to-edge” repair). The MitraClip® mitral valve clip is a metallic implant made of a cobalt chromium alloy and covered with a fabric mesh.

To date, over 100,000 procedures using the MitraClip® valve clip have been performed globally. The MitraClip® procedure has been found to be a very safe procedure and provides a therapeutic option for patients at high surgical risk.

There are, however, some significant limitations to edge to edge clip technology. First, once a mitral valve clip is implanted, removal requires surgical excision. Second, not all patients achieve satisfactory MR reduction at the time of the procedure due to technical or anatomical challenges. Finally, up to one in five patients may experience a return of significant MR or have the need for a repeat intervention MitraClip® procedure.

If patients have recurrent or residual MR after a mitral valve clip procedure, current options for additional treatment are limited. One option would be to place another mitral valve clip, but this is not always possible due to concern for creating mitral stenosis (narrowing of the mitral valve opening that obstructs blood flow from the left atrium to the left ventricle).

Due to the complex nature of mitral valve disease and different patient anatomies, multiple treatment options for MR are necessary for optimum patient care. For patients at high-risk for open heart surgery or in clinical situations when the mitral valve does not meet criteria for treatment with the MitraClip® procedure, favorable options could include a transcatheter mitral valve replacement (TMVR) procedure where a bio-prosthetic mitral valve mounted on an expandable frame is deployed in a defective native mitral valve. For example, one such option, shown in FIG. 5, is a Transcatheter Mitral Valve Implantation (TMVI) system (in particular the Tendyne® TMVI). Such transcatheter “replacement” valves offer complete elimination of mitral regurgitation.

For patients with recurrent/residual MR and/or MS (Mitral Stenosis), previously-inserted mitral valve fixation devices (i.e. MitraClip implants) present a barrier for new TMVI procedures and must be removed before the native valve is replaced with a new implant or bio-prosthetic mitral valve. One such apparatus and method for delivery, repositioning and retrieval of transcatheter prosthetic valves is disclosed in U.S. Patent Publication No. 2018/0028314 A1, which is owned by the Applicant and incorporated herein in its entirety by reference. The devices and methods disclosed in the aforementioned publication are used for the removal of clips and other implanted prostheses from heart valves, while still allowing for the heart valve to be left in a condition suitable for receiving a subsequent transcatheter procedure, such as a prosthetic valve implantation, to treat the pathology.

However, during the removal and subsequent implantation process, the ventricle must be accessed multiple times. With each access, the potential of damaging the myocardium increases the inherent procedural risk to the patient. Accordingly, it would be desirable that such methods and devices for the transcatheter removal of clips and other implanted prostheses from heart valves and subsequent transcatheter procedures, such as prosthetic valve implantation, are able to be performed without the need to access the ventricle multiple times.

### BRIEF SUMMARY OF THE INVENTION

The present disclosure describes devices and methods that can be employed to enable the removal of a mitral valve clip and subsequently deliver a mitral valve implantation while reducing the number of times the myocardium must be accessed during the procedure.

There is currently no clip removal procedure, and therefore multiple procedures would be required to remove the clip and replace the native valve. These procedures may require delivery system to pass through the myocardium multiple times. With each pass, the inherent risk of damaging the myocardium is increased. In order to resolve these problems, it may be desirable to use a device which would allow for the removal of a valve clip and subsequent delivery of a mitral valve implantation during one-time access through the myocardium.

An embodiment of the present invention comprises a system providing for a catheter assembly of a prosthetic mitral valve delivery system to be used with multiple therapeutic devices and remain in place within a patient's anatomy throughout a multi-step therapeutic procedure. The system can comprise, in combination, (1) a clip removal tool or clip management tool (CMT), (2) an implant delivery system having a handle assembly, a valve holding tube, and a catheter assembly, and (3) a modular adapter. The combination can allow the catheter assembly to provide access to a target site for both the clip removal tool and the handle assembly for implant delivery, providing for interchange and use of both devices without having to separately access the target site multiple times to accommodate each separate therapeutic device. The modular adapter can be connected to the catheter assembly to facilitate the use of the clip removal tool with the catheter assembly while maintaining hemostasis.

For example, according to one embodiment of the invention, the modular adapter is a port accessory (PA) device facilitating use of the clip management tool with a catheter assembly for removal of a mitral valve clip. After disconnecting the modular adapter from the catheter assembly, delivery of a mitral valve implantation using the same catheter assembly can be implemented by way of attachment of a valve holding tube and handle assembly to the catheter assembly. The port accessory device can comprise an adapter body. The adapter body can comprise an elongate body having a distal end portion comprising an internal bore extending from the distal opening to the flush chamber interface, a proximal end portion comprising a cavity extending from the flush chamber interface to the proximal opening, and a flush port opening on an annular segment of the proximal end portion. The proximal end portion can comprise an internal cavity having a valve receiving space flush chamber. A valve can be positioned within the valve receiving space of the proximal end portion of the adapter body, preventing fluids such as blood and flushing fluid from flowing out of the cap opening and preventing air ingress into the anatomy of a patient during the procedure. A cap can be connected to a proximal end portion of the housing to selectively seal the valve within the housing of the adapter body. The cap can have an opening aligning with a valve opening or slit, and the internal bore of the adapter body. A flush tube can be selectively connected to the flush port opening on the port accessory device to allow for flushing of the port accessory device and catheter assembly with flush fluid.

The port accessory (PA) device can further include a sealing edge circumscribing a distal tip of the distal end portion of the adapter body and a stop proximal relative to the sealing edge, forming a sealing groove for receiving a seal. The seal can be an o-ring or similar. The distal tip and seal can be inserted into a catheter assembly. By inserting the distal tip with the seal into the catheter assembly, a leak free seal is formed, allowing flush fluids to pass from the port accessory through the catheter assembly.

The present disclosure comprises a method for removal of one or more mitral valve clips with a clip removal tool using a port accessory device to connect to a catheter assembly and subsequent delivery of a mitral valve implantation using the same catheter assembly with a handle assembly in a mitral valve replacement procedure. The method can comprise 1) connecting the port accessory (PA) device to a catheter assembly, 2) connecting the flush port 3-way stopcock to high pressure tubing connected to a pressurized bag, 3) inserting a prepped collapsible dilator balloon through the side port of the catheter assembly, 4) removing the air from the catheter assembly and from the port accessory device by flushing via the PA flush port and then inflating the collapsible dilator balloon to seal the distal end of the catheter assembly, 5) maintaining the proximal PA connection to the flush after preparation of the PA and catheter assembly is complete, 6) gaining access to the left atrium of a heart using standard techniques to position a guidewire in the left atrium, 7) advancing the catheter assembly and the port accessory device over the guidewire and then using the dilator device to dilate an access site and gain access to a left ventricle, 8) advancing the catheter assembly and the port accessory device until a distal end of the delivery sheath of the catheter assembly passes a mitral valve into the left atrium, 9) deflating and retracting the dilator device and guidewire from the side port of the catheter assembly and initiating a slow flush from the flush tube of the port accessory device, 10) aspirating from the catheter assembly side port, 11) inserting a clip management tool through the port accessory device and through the catheter assembly, advancing the clip management tool until the tip of the clip management tool is in the left atrium, 12) retracting the catheter assembly and port accessory device so that the distal end of the delivery sheath of the catheter assembly is in the left ventricle while the clip management tool remains stationary in the left atrium, 13) capturing the mitral valve clip and cutting the clip from the mitral valve with the clip management tool, 14) advancing the catheter assembly and port accessory device so that the distal end of the catheter assembly is in the left atrium while the clip management tool remains stationary, 15) retracting the clip management tool out of the catheter assembly and port accessory device, which remain stationary in the left atrium, 16) inserting a new transition balloon device through the catheter assembly side port, 17) inflating the transition balloon device in the delivery sheath to maintain hemostasis, 18) switching the high pressure tubing flush line from the PA flush port to the catheter assembly side port to ensure a forward flush, 19) removing the port accessory device from the catheter assembly, 20) connecting a valve holding tube loaded with the mitral valve implantation and connected to a handle assembly to the catheter assembly, 21) ensuring a positive flush of the delivery system from the proximal port of the handle assembly, 22) aspirating from the side port of the catheter assembly, 23) deflating and removing the transition balloon device from the side port of the catheter assembly, 24) aspirating from the side port of the catheter assembly, and/or 25) completing the mitral valve implantation delivery procedure per instructions for use. Note, the same method or procedure described herein may also be used to remove multiple clips.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings.

FIG. 1 illustrates a cross-sectional view of a human heart;

FIG. 2A illustrates a top view of a healthy mitral valve;

FIG. 2B illustrates a top view of a diseased mitral valve;

FIG. 3 illustrates a cross-sectional view of a mitral valve repair using a mitral valve clip;

FIG. 4 illustrates a top/atrial perspective view of a mitral valve repaired with a mitral valve clip;

FIG. 5 illustrates a cross-sectional view of the left atrium and left ventricle of a heart with a prosthetic mitral valve (PMV) deployed therein;

FIG. 6 illustrates an embodiment of an assembled delivery system consisting of a catheter assembly, valve holding tube, and handle assembly);

FIG. 7 illustrates an exploded view of the assembled delivery system of FIG. 6;

FIGS. 8A-8B illustrate an embodiment of the port accessory device of the present disclosure assembled on an embodiment of a catheter assembly;

FIGS. 9A and 9B illustrate exploded views of the assembled catheter assembly with a port accessory device of FIGS. 8A-8B;

FIG. 10 illustrates a cross-sectional view of an assembled port accessory device and catheter assembly;

FIG. 11 illustrates a perspective view of an adapter body;

FIG. 12 illustrates a rear perspective view of an adapter body;

FIG. 13 illustrates a cross-sectional view of an adapter body;

FIG. 14A illustrates a side view of a valve;

FIG. 14B illustrates a bottom view of a valve;

FIG. 15 illustrates a side view of an assembled port accessory device;

FIG. 16 illustrates an exploded view of the port accessory device of FIG. 15; and

FIG. 17 illustrates a flowchart of an example method for removal of a mitral valve clip and subsequent delivery of a mitral valve implantation.

### DETAILED DESCRIPTION OF SOME EXAMPLE EMBODIMENTS

Embodiments of the present invention generally relate to the removal repair device and delivery of mitral valve replacement implantations. More particularly, at least some embodiments of the invention relate to devices and methods for removal of a mitral valve clip and subsequent delivery of a mitral valve implant requiring only one-time access to the left ventricle.

### A. Cardiac Physiology

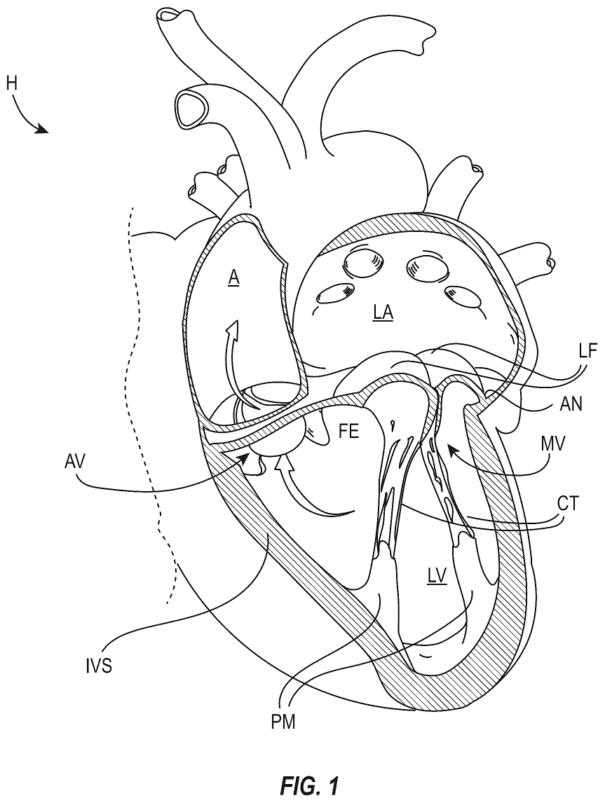

The left ventricle (LV) of a normal heart H in systole is illustrated in FIG. 1. The left ventricle (LV) is contracting and blood flows outwardly through the tricuspid aortic valve (AV) in the direction of the arrows. Back flow of blood or “regurgitation” through the mitral valve (MV) is prevented since the healthy mitral valve is configured as a “check valve” which prevents back flow when pressure in the left ventricle is higher than that in the left atrium (LA). The mitral valve (MV) comprises a pair of leaflets having free edges (FE) which meet evenly to close, as illustrated in FIG. 1. The opposite ends of the leaflets (LF) are attached to the surrounding heart structure along an annular region referred to as the annulus (AN). The free edges (FE) of the leaflets (LF) are secured to the lower portions of the left ventricle LV through chordae tendineae (CT) (referred to hereinafter as the chordae) which include a plurality of branching tendons secured over the lower surfaces of each of the valve leaflets (LF). The chordae tendineae (CT) in turn, are attached to the papillary muscles (PM) which extend upwardly from the lower portions of the left ventricle and intraventricular septum (IVS).

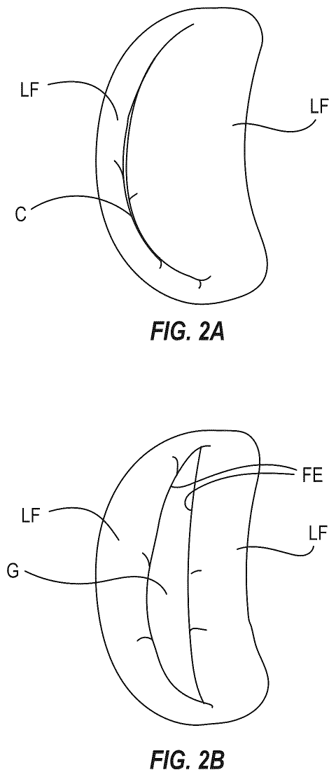

A number of structural defects in the heart can cause mitral valve regurgitation. Regurgitation occurs when the valve leaflets do not close properly allowing leakage from the ventricle into the atrium. As shown in FIG. 2A, the free edges of the anterior and posterior leaflets normally meet along a line of coaptation (C). An example of a defect causing regurgitation is shown in FIG. 2B. Here an enlargement of the heart causes the mitral annulus to become enlarged, making it impossible for the free edges (FE) to meet during systole. This results in a gap (G) which allows blood to leak through the valve during ventricular systole. Ruptured or elongated chordae can also cause a valve leaflet to prolapse since inadequate tension is transmitted to the leaflet via the chordae. While the other leaflet maintains a normal profile, the two valve leaflets do not properly meet and leakage from the left ventricle into the left atrium will occur. Such regurgitation can also occur in patients who have suffered ischemic heart disease where the left ventricle does not contract sufficiently to effect proper closure.

### B. Overview of Mitral Valve Fixation Technology

Fixation devices are used for grasping, approximating and fixating tissues such as valve leaflets to treat cardiac valve regurgitation, particularly mitral valve regurgitation. The fixation devices may also provide features that allow repositioning and removal of the device prior to deployment, if so desired, particularly in areas where removal may be hindered by anatomical features such as chordae tendineae CT. Such removal would allow the physician to re-approach the valve in a new manner if so desired.

When describing the devices of the invention herein, “proximal” shall mean the direction toward the end of the device to be manipulated by the user outside the patient's body, and “distal” shall mean the direction toward the working end of the device that is positioned at the treatment site and away from the user. With respect to the mitral valve, distal shall refer to the atrial or upstream side of the valve leaflets, and proximal shall refer to the ventricular or downstream side of the valve leaflets.

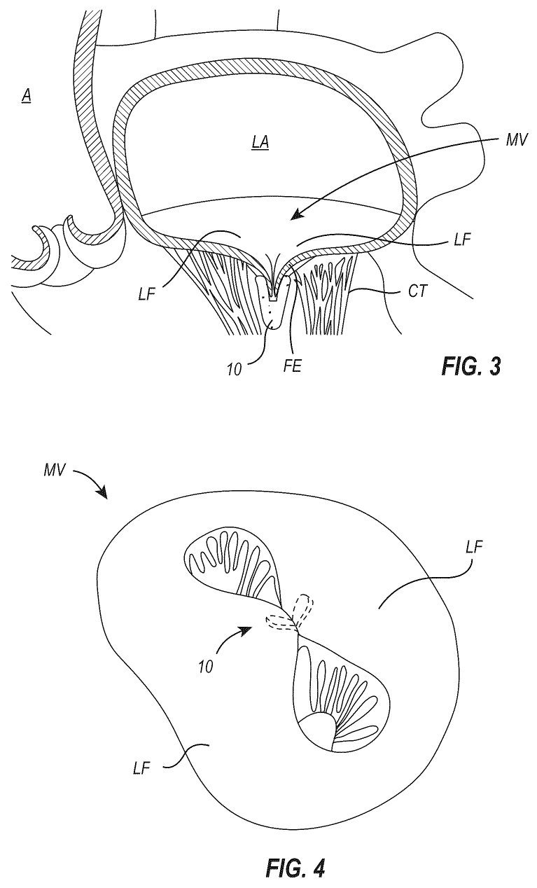

FIG. 3 and FIG. 4 show an example of a fixation device 10 as implanted on the mitral valve MV by grasping the leaflets LF. The mitral valve fixation device 10 may be the MitraClip® or a functionally equivalent device. The mitral valve may be accessed either surgically or by using endovascular techniques, and either by a retrograde approach through the ventricle or by an antegrade approach through the atrium.

In some situations, it may be desired to remove the fixation device 10. Such removal may be desired to attempt to achieve better valve function with a bio-prosthetic mitral valve implant such as the Tendyne® TMVI. A tool for fixation device 10 removal can include a clip removal tool or clip management tool (CMT). Generally, a clip removal tool comprises a series of catheters which are positioned within a chamber of the heart adjacent to leaflets of a valve. Once in position, a cutting element can be deployed through a catheter to the tissue of a valve leaflet, which can then engage the cutting element against a valve leaflet and excise the fixation device from the valve leaflet.

Transcatheter tools and procedures for separating fixation devices from a mitral valve may include the use of endovascular methods and medical devices, such as for example, those described in U.S. Publication No. 2018/0008268 A1; U.S. Publication No. 2014/0228871 A1; U.S. Publication No. 2015/0257883 A1; U.S. Publication No. 2014/0135799 A1; U.S. Pat. No. 8,500,768 B2; U.S. Publication No. 2017/040977 and U.S. Publication No. 2018/0028314 A1 (the “'8314 Publication”), each of which are owned by Applicant and incorporated herein in their entirety by reference.

### C. Description of the Port Accessory Device

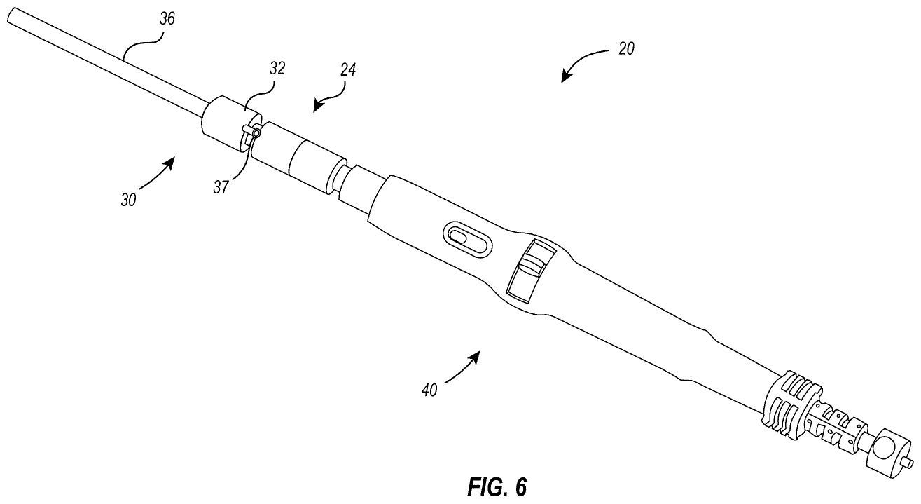

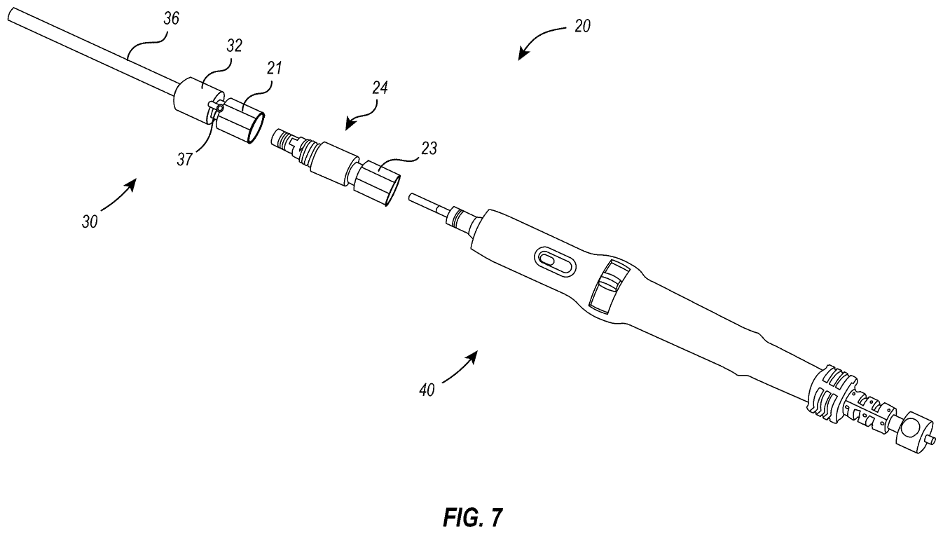

A delivery system can be used to deliver and deploy a prosthetic heart valve within the heart, such as, for example, a prosthetic mitral valve as disclosed in the incorporated references. FIGS. 6-7 illustrate an exemplary embodiment of a delivery system 20 for delivery of a prosthetic heart valve within the heart as disclosed in the '8314 Publication. The delivery system 20 includes a catheter assembly 30, a handle assembly 40 removably couplable to the valve holding tube 24 and a valve holding tube 24 removably couplable to both the handle assembly 40. The catheter assembly 30 includes a hub 32, a delivery sheath 36 and a connector 21. The delivery sheath 36 defines a lumen (not shown) into which a prosthetic valve (not shown) pre-disposed within a valve holding tube 24 can be moved during delivery of the prosthetic valve. The hub 32 is disposed proximal to the delivery sheath 36 and defines an interior region through which the prosthetic valve is first introduced prior to insertion into the lumen of the delivery sheath 36. In use, the hub 32 remains outside of the heart and can provide access to the lumen of the delivery sheath 36 when it is inserted into the heart.

The hub 32 also includes a side port 37 through which various devices, such as for example, a dilator device (not shown) can be inserted and used during removal of a mitral valve fixation device 10, such as a mitral valve clip, and during the delivery of a prosthetic heart valve, which is described in more detail herein. Other dilator devices known in the art to be compatible with the methods and devices described herein may be used. The side port 37 can also be used to receive a guidewire therethrough. For example, a guidewire can be threaded through the distal end of the delivery sheath 36, into the interior of the hub 32, and out through the side port 37. Side port 37 can also be used to flush and deair the system.

The valve holding tube 24 is removably couplable to the catheter assembly 30 and handle assembly 40 by connectors 21, 23. Connectors 21, 23 can be loosened to remove the valve holding tube 24 and tightened to attach the port accessory device 100 of the present invention to the catheter assembly 30.

In general, example embodiments of the invention comprise a port accessory device 100 which can be coupled to a catheter assembly 30 of a delivery system 20 to facilitate removal of one or more fixation devices (e.g., a fixation device 10). The fixation device 10 may be (but is not limited to) a mitral valve clip, implanted or deployed in a mitral valve repair procedure. The procedure can be completed during a one-time access or single passage through a patient's myocardium to the left ventricle. The port accessory device 100 can eliminate the need to puncture the myocardium multiple times to access the left ventricle, thereby reducing inherent risks associated with valve repair and replacement procedures.

The port accessory device 100 can be a modular adapter that has a distal connector, which is configurable to be compatible with the corresponding connector of a catheter assembly 30 of an existing transcatheter mitral valve replacement (TMVR) delivery system 20, such as for example, the device of the '8314 Publication. The modular adaptability of the port accessory device 100 can allow the port accessory device 100 to be selectively attached to the proximal end connector 21 of a catheter assembly 30 of an existing TMVR delivery system 20. The selective connectivity of the port accessory device 100 to the catheter assembly 30 allows the catheter assembly 30 to remain in place throughout a procedure which may involve multiple steps. The catheter assembly 30 can remain in place and be used with other therapeutic devices, such as a clip removal tool or a handle assembly 40 with valve loading tube 24, all while allowing the hemostasis to be controlled during the use and interchanging of multiple therapeutic devices. While the embodiments illustrated and described herein are specific to use with the device of the '8314 Publication, the principles and concepts discussed herein can be adapted to provide similar modular adaptors compatible with other therapeutic devices and procedures.

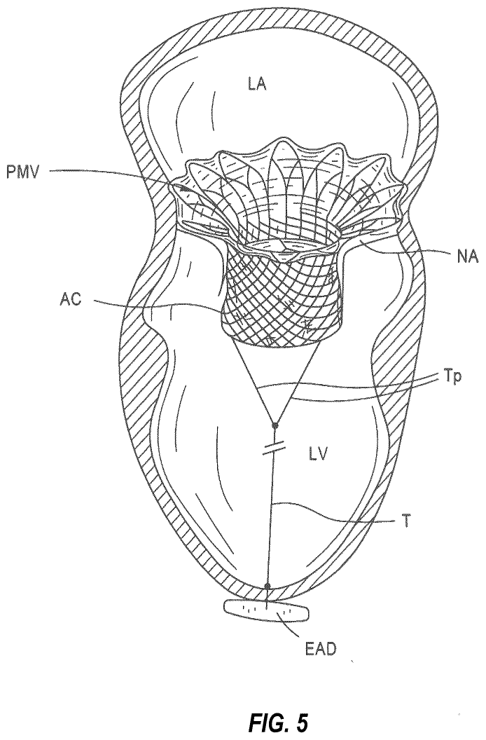

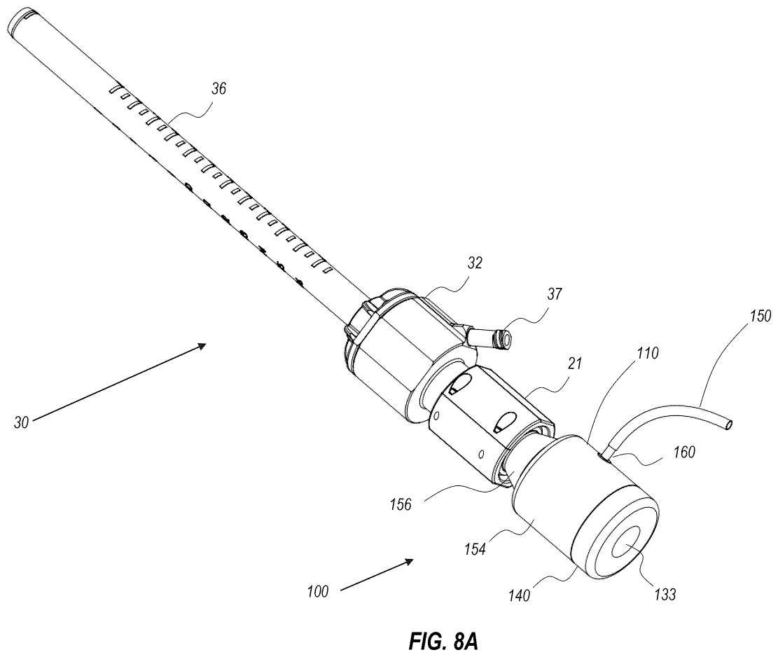

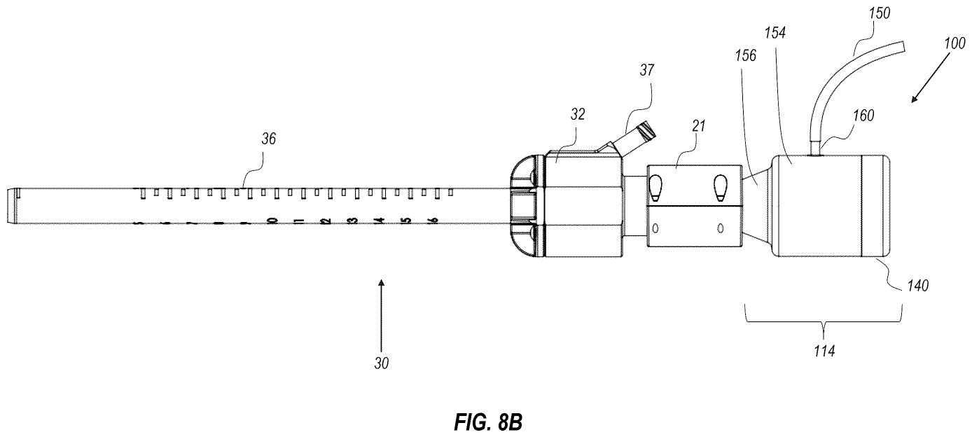

Turning now to FIGS. 8A, 8B, 9A, 9B, and 10, a port accessory device 100 can be connected to a catheter assembly 30, or to a similarly configured catheter assembly, to provide for one-time passage through the myocardium for removal of a mitral valve fixation device 10 and subsequent delivery of a mitral valve implantation PMV (FIG. 5) in a mitral valve replacement procedure. The port accessory device 100 can be a modular adapter configured to be selectively attached to multiple therapeutic devices during a multi-step therapeutic procedure. The port accessory device 100 may also be referred to herein as “PA 100” or “PA device 100”.

A system for fixation device removal and subsequent implantation of a PMV or other implantable device can comprise (1) a clip removal tool or CMT (not shown), (2) an implantation delivery system 20 having a catheter assembly 30, a handle assembly 40 and a valve holding tube 24, and (3) a modular adapter, such as port accessory device 100. The handle assembly 40 and the valve holding tube 24 can be removed from the delivery system 20. The modular adapter can then be configured on the proximal end portion of a catheter assembly 30 and then the distal end of the delivery sheath 36 of a catheter assembly 30 can be positioned at a target site, such as within a heart chamber. Once the delivery sheath 36 is positioned at the target site, the clip removal tool can be delivered to the target site by insertion of the clip removal tool through the port accessory device 100 and through the catheter assembly 30. Once the fixation device 10 is removed from the valve leaflets, the clip removal tool can be removed from the catheter assembly 30 and the attached port accessory device 100. The catheter assembly 30 can remain in the heart chamber while the port accessory device 100 is removed from the catheter assembly 30 and replaced with a valve holding tube 24 connected to a handle assembly 40 to form a delivery device 20 (see FIGS. 6-7). The delivery system 20 can then deliver a PMV through the same catheter assembly 30. The port accessory device 100 allows for use and interchange of various therapeutic devices with a single catheter assembly 30, thereby requiring only a one-time passage of the catheter assembly 30 through the myocardium.

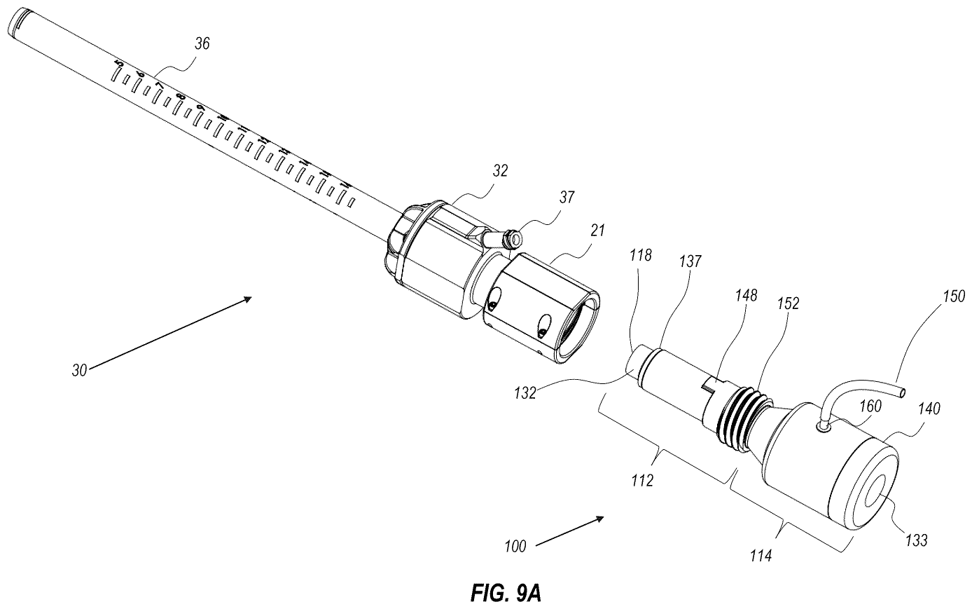

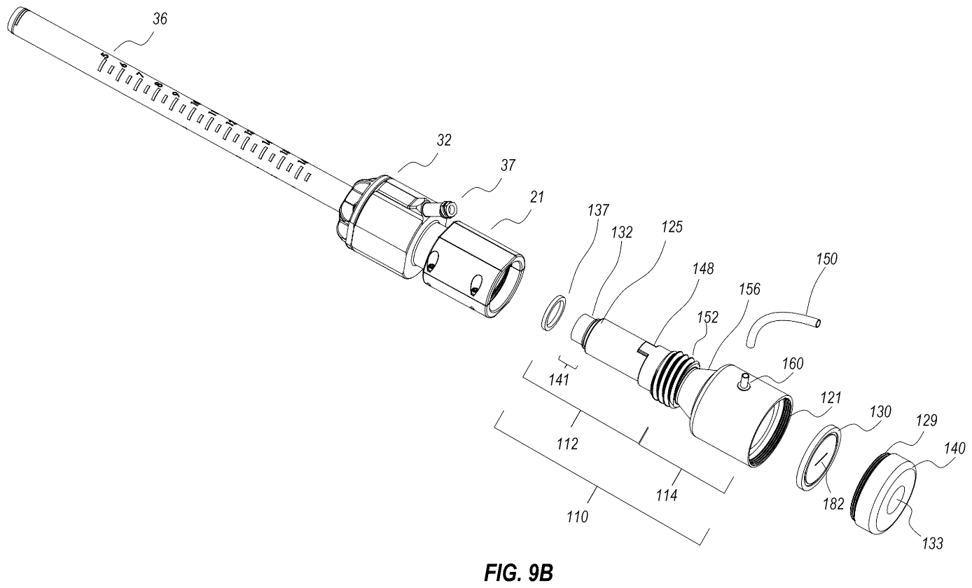

Referring to FIGS. 8A-8B, 9A-9B, 10, and 15-16, the fully assembled PA device 100 can comprise an adapter body 110 having a distal end portion 112 and a proximal end portion 114. A cap 140 can be configured to be selectively coupled to the proximal end portion 114. The PA device 100 further includes a valve 130 (see FIGS. 9B, 10, 14A, 14B, 16) disposed within the cavity 164 of the proximal end portion 114 of the adapter body 110. The cap 140 is selectively connected to the proximal end portion 114 of the adapter body 110 to secure the valve 130 within the adapter body 110. The proximal end portion 114 also includes a port 160 to which a flush port tube 150 can be attached to the adapter body 110. In some embodiments, the port 160 can have a feature to secure the attachment of the flush tube 150 to the adapter body 110. In some embodiments, the port 160 can be a standard luer fitting. It may be advantageous for the port 160 to be positioned on top of the port accessory device 100 when in use in order to monitor and control flushing of the PA device 100.

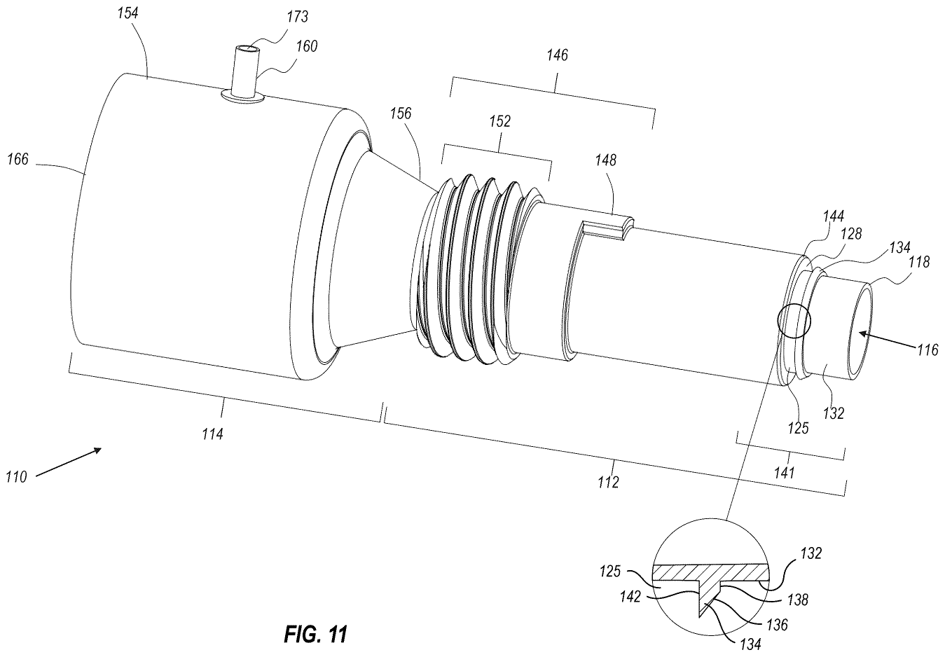

FIGS. 11-13 illustrate various perspective views of an embodiment of an adapter body 110 of a PA device 100. The adapter body 110 comprises an elongate body having a distal end portion 112 and a proximal end portion 114.

An internal bore 116 extends the length of the distal end portion 112 between a distal opening 118 and a flush chamber interface 122. The flush chamber interface 122 can define the terminus of the internal bore 116. The distal end of the distal end portion 112 can comprise a distal tip 141. The start of the distal tip 141 is defined by the stop 128 and extends to the distal opening 118. The stop 128 can have a larger diameter than the diameter of the distal tip 141 in order to provide a sealing effect when the PA device 100 is inserted into a hub 32 of the catheter assembly 30. As such, an external side of the stop 128 has a greater diameter than an external side 132 of the distal tip 141.

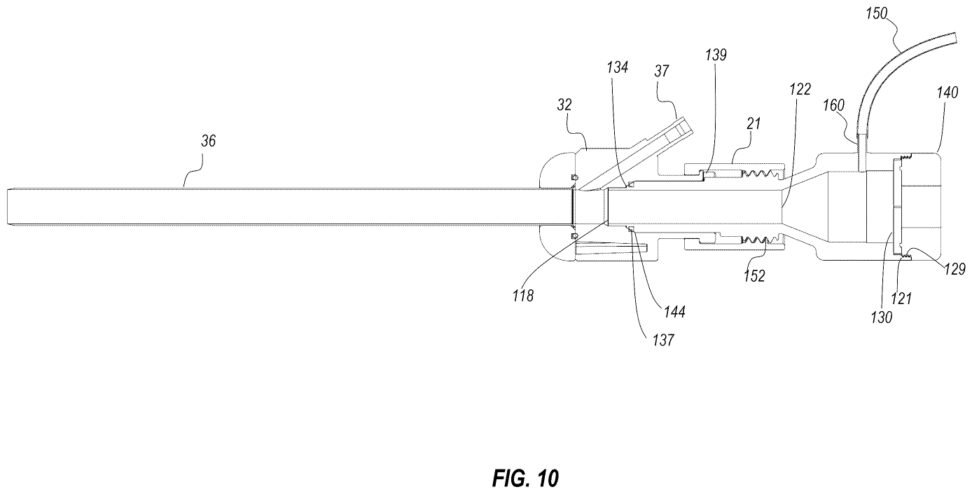

The external side 132 of the distal tip 141 can have a sealing groove 125 around its circumference. The annular recess or sealing groove 125 formed between the edge 134 and the stop 128 allows for a seal 137 to be positioned and secured within the sealing groove 125. The edge 134 can have first protruding side 138, a second protruding side 142, and an angular side 136 (connecting the first protruding side 138 and the second protruding side 142) to prevent any interference when connecting with catheter assembly 30 and provide a better seal. The seal 137 can be an o-ring having a square, rectangular, or circular cross section, dependent on the shape of the sealing groove 125. The seal 137 can be made of any flexible, waterproof material such as silicone, polyurethane or other suitable material. The seal 137 together with the edge 134, stop 128, and sealing groove 125 creates a leak-proof seal when the distal end portion 112 of the PA device 100 is inserted into the catheter assembly 30, as shown in FIG. 10. The leak-proof seal may be advantageous in preventing potential air ingress and the escape of blood or flush fluid delivered to the PA device 100 via the flush tube 150. The leak-proof seal allows for hemostasis to be controlled during use and interchange of multiple therapeutic devices throughout a multi-step therapeutic procedure. The leak-proof seal formed by compressing the seal 137 against the catheter assembly 30 may also increase the efficiency of the flushing process.

The distal end portion 112 can also have a connecting portion 146. The connecting portion 146 can generally comprise one or more features configured to provide a secure connection with the hub 32 when the PA device 100 is inserted into the catheter assembly 30. The adapter body 110 of FIGS. 9B, 11-13, 14A and 14B includes a keyed fit structure 148 and a threaded portion 152 comprising a plurality of threads. In some embodiments, the adapter body 110 further includes a connection portion 143 disposed between the threaded portion 152 and the external side 144 of the stop 128. The keyed fit structure 148 can be configured to mate with corresponding features inside of a hub 32 in order to ensure the proper orientation of the PA device 100 when inserted into the catheter assembly 30. The threaded portion 152 can be configured to mate with corresponding threading in the catheter assembly 30, or connector 21 or a similar device.

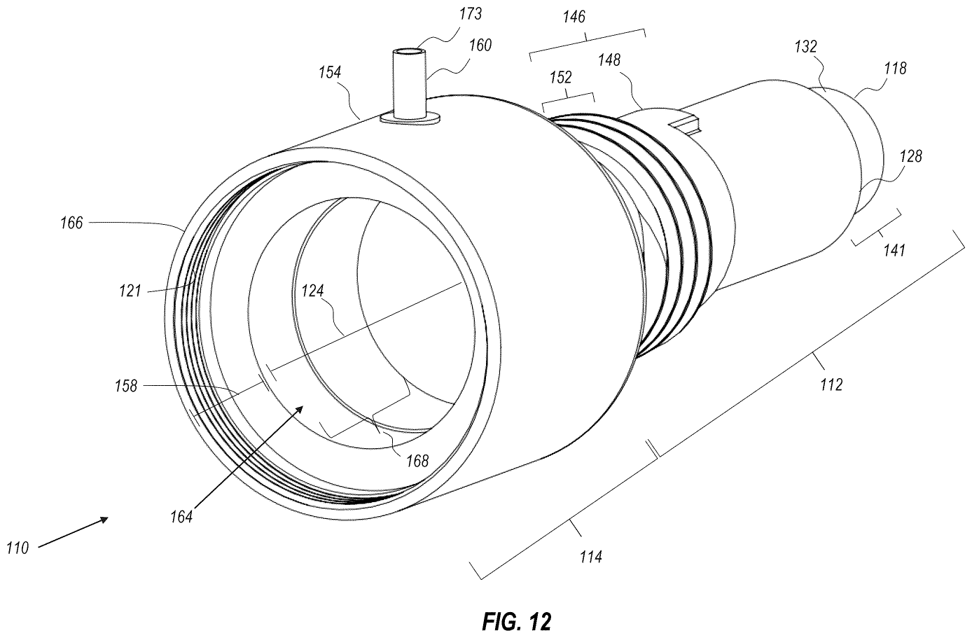

As illustrated in FIGS. 11-12, the proximal end portion 114 of the adapter body 110 comprises annular segments 154, 156. The segments may be uniform annular segments 154 having a uniform outer diameter, while others can be graduated segments 156 having an outer diameter which increases in size from one end of the segment to the other. In another embodiment, the proximal end portion 114 can have a uniform diameter throughout, while in other embodiments, the proximal end portion 114 may take the form of any shape suitable to house the size and shape of valve 130.

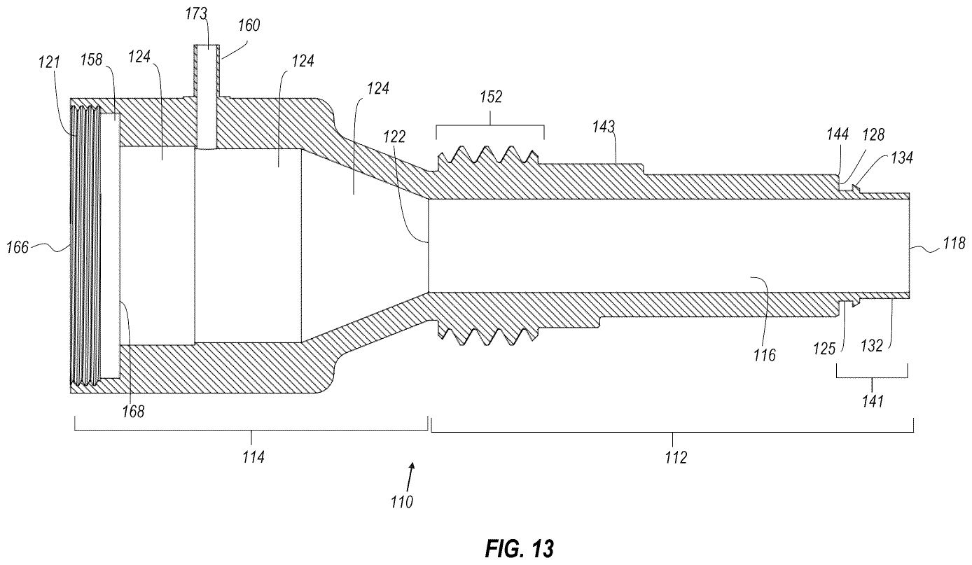

Annular segments 154, 156 of the proximal end portion 114 can combine to form a flush chamber 124 proximal to the external connecting portion 146. The flush chamber 124 further includes a flush port 160 having an opening 173. A flush port tube 150 (shown in FIGS. 8B, 9B, 15-16) can be connected to the flush port 160 in order to provide a fluid flush to the PA device 100 and catheter assembly 30 during removal of a mitral valve repair device. The internal bore 116 can terminate at the flush chamber interface 122, as shown in FIG. 13.

A portion of the cavity 164 forms the flush chamber 124, while another portion of the cavity 164, proximal relative to the flush chamber 124, comprises a valve receiving space 158. The valve receiving space 158 can be configured to receive and secure a valve 130.

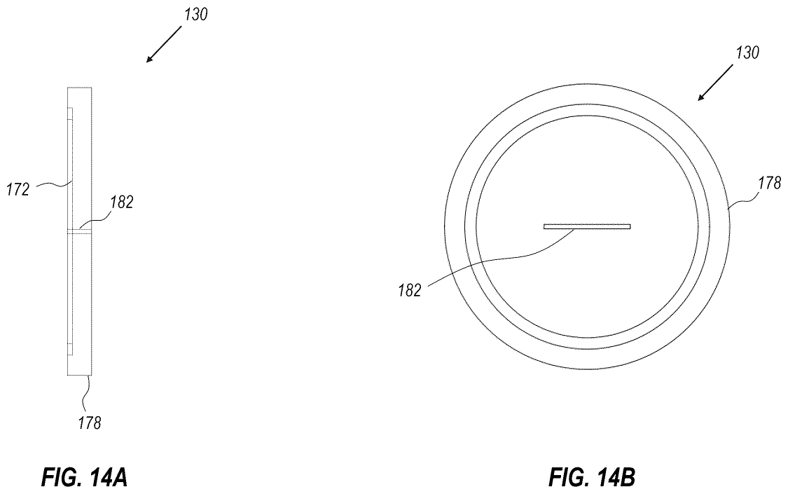

A valve 130, illustrated in FIGS. 14A and 14B, can be inserted through the proximal opening 166 and positioned within the valve receiving space 158. As illustrated, the valve 130 includes a base edge 178, a concave portion 172, and a valve slit 182. FIGS. 14A and 14B merely illustrate an example of an embodiment of a valve 130. In some embodiments, the valve 130 can have a complementary shape to the internal structure 168 of the valve receiving space 158, which may advantageously prevent the valve 130 from advancing further into the flush chamber 124 which may cause leakage. The valve 130 may be constructed of a flexible material such as silicone, polyurethane or other suitable material with different slit patterns to balance the seal and friction. The valve 130 can form a seal on and allow for the passage of a clip removal tool such as a mitral clip management tool, while preventing fluid leakage and air ingress through the cap opening 133 of the PA device 100. In some embodiments, the valve 130 can comprise two valves combined and placed within the valve receiving space 158 to form a leak-proof seal and allow for the insertion and removal of a clip management tool.

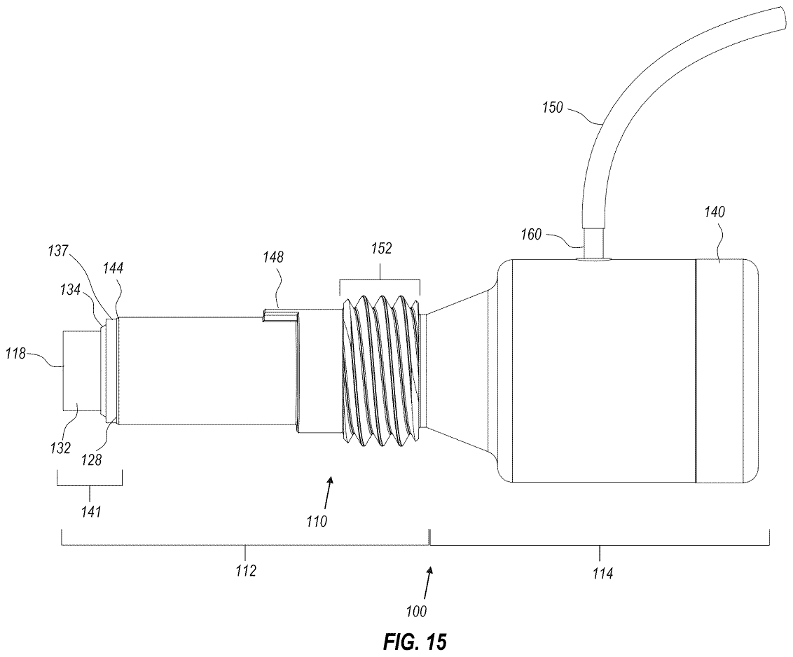

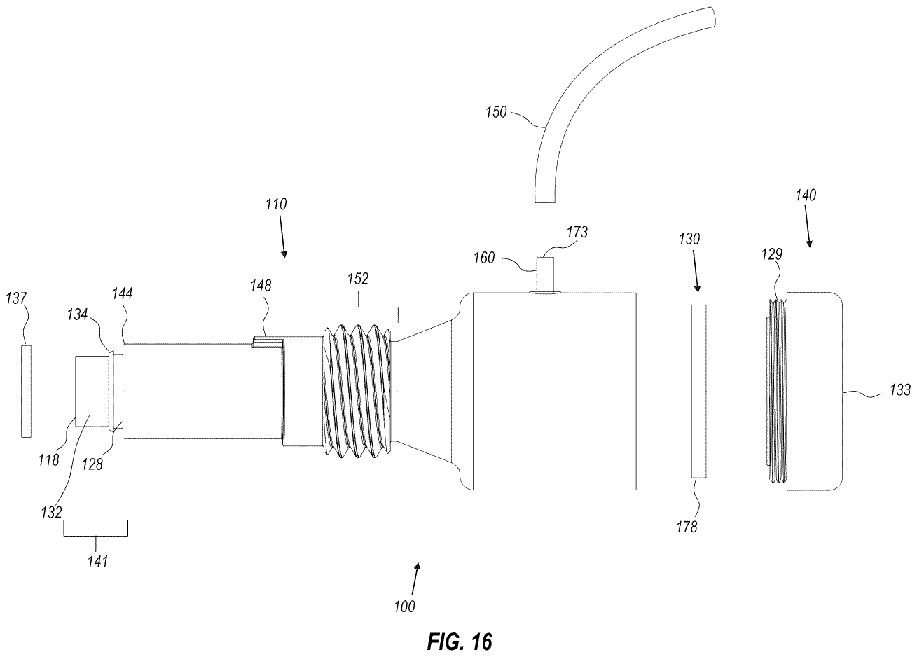

FIGS. 15-16 illustrate the modular adapter or port accessory device 100 fully assembled. FIGS. 15 and 16 illustrate the port accessory device 100 having the adapter body 110 of FIGS. 11-13, 14A and 14B. The proximal end portion 114 of the adapter body 110 can have an internally threaded portion 121 configured to mate with corresponding cap threads 129 on a cap 140. A portion of the valve receiving space 158 adjacent to the proximal opening 166 can include internally threaded portion 121 configured to mate with corresponding threads on the cap 140. The cap 140 can include an opening 133 which can be sized to correspond with and align with the valve slit 182 and the internal bore 116 of the distal end portion 112 of the adapter body 110. In some embodiments, the cap 140 can be bonded to the proximal end portion 114 of the adapter body 110 with adhesive or other technologies, for example ultrasonic welding. When secured to the adapter body 110, the cap 140 can compress the base edge 178 of the valve 130 against the internal structure 168 of the valve receiving space 158 to form a leak free seal around the valve 130 and secure the valve 130 in the correct place within the valve receiving space 158.

The port accessory 100 can have various lengths to accommodate various different procedures for removal of mitral valve repair devices. For example, in some embodiments, the port accessory 100 can have a length of between 5 cm and 20 cm.

In some embodiments, the delivery sheath 36 of the catheter assembly 30 can have a length of about 12 cm to about 38 cm. In some embodiments, the delivery sheath 36 can have a length of about 50 cm to about 150 cm.

In some embodiments, the prosthetic heart valve or mitral valve replacement (e.g., mitral valve) can be delivered apically, i.e., delivered through the apex of the left ventricle of the heart, using a delivery system, such as for example, delivery system 20. With such apical access, the heart and pericardial space can be accessed by intercostal delivery. In this case, the delivery sheath 36 can have a length of, for example, 10 cm to 40 cm.

### D. Method for Removal of a Mitral Fixation Device and Subsequent Implantation of a Transcatheter Mitral Valve Prosthesis Utilizing the Port Accessory Device

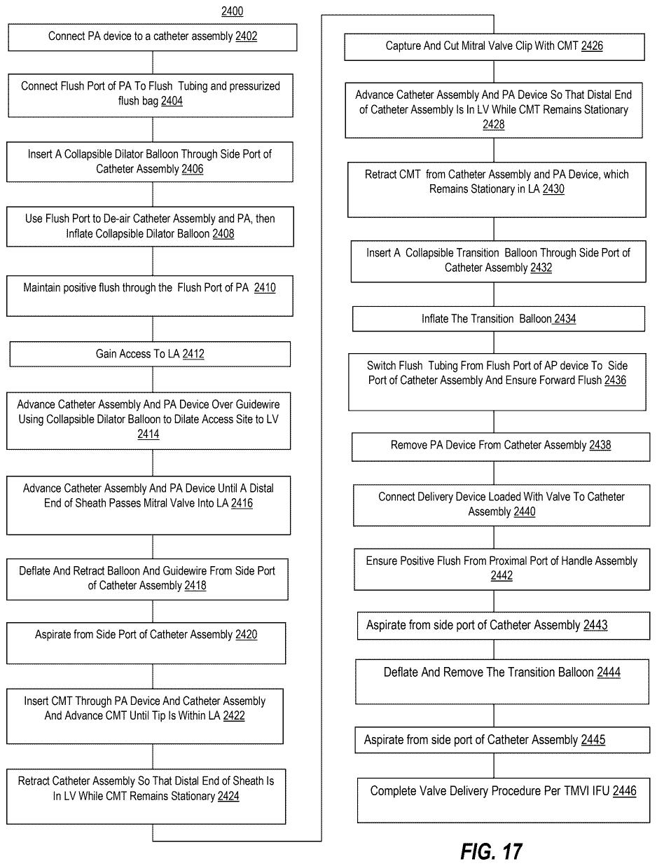

Turning now to FIG. 17, FIG. 17 illustrates a flowchart of an example method 2400 of removal of a mitral valve clip and subsequently delivery of a mitral valve implantation, which may be performed using the PA device 100. The method 2400 includes connecting a PA device (e.g., PA device 100) to a catheter assembly (e.g., catheter assembly 30) (act 2402) and connecting a flush line to the flush port of the PA device 100 (e.g., port 160) (act 2404). In some embodiments, the flush port tube is a high-pressure tubing that is connected to a pressurized bag. The method 2400 also includes inserting a collapsible dilator balloon through a side port (e.g., side port 37) (act 2406). The method 2400 also includes de-airing the catheter assembly and PA 100 by flushing through flush port and inflating the collapsible dilator balloon (act 2408). Positive flush on the PA device 100 is maintained (act 2410), and access to a left atrium is gained (act 2412). Thereafter, the catheter assembly and the PA device are advanced over a guidewire using the collapsible dilator balloon to dilate an access site to a left ventricle (act 2414). The catheter assembly and the PA device are further advanced until a distal end of a delivery sheath (e.g., delivery sheath 36) of the catheter assembly passes a mitral valve into the left atrium (act 2416). The collapsible dilator balloon is then deflated, and the collapsible dilator balloon and the guidewire are retracted from the side port of the catheter assembly (act 2418).

In some embodiments, the method 2400 further includes aspirating from side port of catheter assembly (2420). A CMT is then inserted through the PA device and catheter assembly and advanced until a tip of the CMT is within the left atrium (act 2422). After that, the catheter assembly is retracted, so that a distal end of the sheath is in the left ventricle while CMT remains stationary (act 2424). A mitral valve clip is then captured and cut from the mitral valve with CMT (act 2426). Next, the catheter assembly and the PA device are advanced, so that the distal end of the catheter assembly is in the left atrium while the CMT remains stationary (act 2428). The CMT is then retracted while the catheter assembly and port assembly remains stationary (act 2430). A collapsible transition balloon is inserted through the side port of the catheter assembly (act 2432) and then inflated (act 2434). The flush port tubing is then switched from the flush port of the PA device to a sheath side port of the catheter assembly, and forward flush is ensured (act 2436). Thereafter, the PA device is removed from the catheter assembly (act 2438). Next, a delivery device loaded with a valve is connected to the catheter assembly (act 2440), and a positive flush is ensured from a proximal port of a handle assembly (act 2442). The side port of the catheter assembly is aspirated (act 2443). The transition balloon is then deflated and removed (act 2444). The side port of the catheter assembly is aspirated a second time (act 2445). Finally, a valve delivery procedure is completed per TMVI IFU steps (act 2446).

PA device 100, as described above, can be incorporated into a procedure involving removal of a previously placed fixation device 10, such as a mitral valve clip, from a mitral valve, and subsequently replacing the fixation device with a transcatheter mitral valve replacement (TMVR), in order to reduce the number of times the clip management tool and delivery system 20 must pass through the myocardium. The reduction in the number of passes through the myocardium can decrease the inherent procedural risk of damaging the myocardium.

The PA device 100 is necessary for a successful one-time access of the left ventricle LV in a mitral clip removal and transcatheter mitral valve replacement procedure. To begin the procedure, the first step is to gain access to the left atrium LA of a patient's heart using standard access techniques. The guidewire can be left in the left atrium LA to later guide the catheter assembly 30 and inflatable dilator through the myocardium.

The PA device 100 can be connected to the catheter assembly 30 by aligning the keyed fit structure 148 with complementary receiving structure 139 (see FIG. 10) on the hub 32. The distal end portion 112 of the PA device 100 is inserted into the catheter assembly 30 and selectively secured to the catheter assembly 30. The seal 137 of the distal end portion 112 provides a leak free seal when the distal tip 141 is positioned within the catheter assembly 30. Air is then removed from the delivery catheter assembly 30 and the PA device 100 by initiating a flush of the PA device 100 by introducing flush fluid to the flush chamber 124 of the PA device 100 via a flush tube 150 connected to the flush port 160.

A dilator device, such as a dilator balloon, can be inserted into the catheter assembly 30 via the side port 37 and inflated to between approximately 3 and approximately 8 ATM inside of the delivery sheath 36.

The catheter assembly 30 and PA device 100 can be advanced over the guidewire, and then the dilator device can be used to dilate the access site and gain access into the left ventricle LV. Once in the left ventricle LV, the catheter assembly 30 and PA device 100 can be advanced until the distal end of the delivery sheath 36 passes the mitral valve MV. The dilator device can be deflated, and the dilator device and the guidewire can be retracted from the side port 37 and a slow flush of the PA device 100 and catheter assembly 30 can be initiated.

Next, a clip management tool can be inserted into the PA device 100 through the opening 133 in the cap 140, through the valve 130, and through the internal bore 116 of the PA device 100 and into and through the hub 32 and delivery sheath 36. The clip management tool can be advanced beyond the distal end of the delivery sheath 36 until the tip of the clip management tool is within the left atrium LA. Once the tip of the clip management tool is in the left atrium LA, the clip management tool can be held in a stationary position while the catheter assembly 30 can be retracted so that the distal end of the delivery sheath 36 is in the left ventricle LV. Then, the clip management tool can be used to capture the mitral valve clip and cut the clip from the mitral valve with the clip management tool.

Next, the catheter assembly 30 can be advanced over the clip management tool while the clip management tool is held stationary, so that the distal end of the delivery sheath 36 can be positioned in the left atrium LA. The clip management tool can be retracted from the catheter assembly 30 and the PA device 100 while maintaining positive flush of the PA device 100 and the catheter assembly 30 via the flush tube 150 and flush port 160.

Once the clip management tool is removed from the catheter assembly 30 and PA device 100, the transition balloon device can be inserted into the side port 37 of the catheter assembly 30 and inflated to between approximately 3 and approximately 8 ATM. Inflation of the balloon device can prevent blood leakage when the PA device 100 is removed from the catheter assembly 30. The PA device 100 can then be removed from the catheter assembly 30.

Next, a valve holding tube 24 pre-loaded with a replacement valve and the handle assembly 40 can be connected to the catheter assembly 30 in a manner similar to the connection used to previously connect the PA device 100 to the catheter assembly 30, to form the delivery system 20. Air is then further removed from the delivery system 20 and then the transition balloon device is deflated and removed from the catheter assembly 30 through side port 37. The TMVR delivery procedure can proceed per the standard instructions for use (IFU).

While the embodiments and methods illustrated and described herein are specific to use with the device of the '8314 Publication, the principles and concepts discussed herein can be adapted to provide similar modular adaptors compatible with other therapeutic devices and procedures.

Embodiments of the invention, such as the examples disclosed herein, may be beneficial in a variety of respects. For example, and as will be apparent from the present disclosure, one or more embodiments of the invention may provide one or more advantageous and unexpected effects, in any combination, some examples of which are set forth below. It should be noted that such effects are neither intended, nor should be construed, to limit the scope of the claimed invention in any way. It should further be noted that nothing herein should be construed as constituting an essential or indispensable element of any invention or embodiment. Rather, various aspects of the disclosed embodiments may be combined in a variety of ways so as to define yet further embodiments. Such further embodiments are considered as being within the scope of this disclosure. As well, none of the embodiments embraced within the scope of this disclosure should be construed as resolving, or being limited to the resolution of, any particular problem(s). Nor should any such embodiments be construed to implement, or be limited to implementation of, any particular technical effect(s) or solution(s). Finally, it is not required that any embodiment implement any of the advantageous and unexpected effects disclosed herein.

### E. Further Example Embodiments

Following are some further example embodiments of the invention. These are presented only by way of example and are not intended to limit the scope of the invention in any way.

Embodiment 1. A system for removal of a mitral valve clip and subsequent delivery of a mitral valve implantation in a mitral valve repair procedure, the system comprising, in combination, a clip removal tool, a delivery system having a catheter assembly and handle assembly, and a modular adapter, the modular adapter comprising, an adapter body, the adapter body comprising an elongate body having a proximal end portion and a distal end portion, wherein the proximal end portion comprises an internal cavity extending the length of the proximal end portion between a proximal opening and flush chamber interface, and wherein the distal end portion comprises a distal tip and an internal bore extending the length of the distal end portion between a distal opening and a flush chamber interface, a port opening on the proximal end portion, a seal positioned on the distal tip, a valve with different slit patterns positioned within a receiving space of the proximal end portion of the adapter body, a cap connected to a proximal end portion of the adapter body to selectively secure the valve within the proximal end portion of the adapter body, wherein the cap has a cap opening aligning with a valve opening and the internal bore of the adapter body, and a flush tube selectively connected to the port opening, wherein the combination allows the catheter assembly to provide access to a target site for both the clip removal tool and a delivery system, allowing for interchangeability and use of both devices without having to separately access the target site multiple times.

Embodiment 2. The system as recited in embodiment 1, wherein the adapter body has a connecting portion configured to selectively secure the modular adapter to the catheter assembly.

Embodiment 3. The system as recited in any of embodiments 1-2, wherein an edge and a stop form a sealing groove therebetween, and the seal is positioned within the sealing groove to provide a leak-free seal when selectively connected to the catheter assembly.

Embodiment 4. The system as recited in any of embodiments 1-3, wherein the connecting portion comprises a plurality of threads configured to interface with the catheter assembly.

Embodiment 5. The system as recited in any of embodiments 1-4, wherein the connecting portion further comprises a keyed fit structure.

Embodiment 6. The system as recited in any of embodiments 1-5, wherein the connecting portion is located proximally relative to the distal tip.

Embodiment 7. The system as recited in any of embodiments 1-6, wherein is the distal tip is recessed to allow the distal tip to be inserted into an opening of larger diameter relative to the diameter of the distal tip, thereby causing the seal to be compressed within the catheter assembly.

Embodiment 8. The system as recited in any of embodiments 1-7, wherein the valve is positioned proximal to the flush port within the receiving space of the adapter body so as not to obscure the flow of flush fluid from the flush port into the flush chamber of the adapter body.

Embodiment 9. The system as recited in any of embodiments 1-8, wherein the keyed fit structure is configured to align with and engage a hub of the catheter assembly.

Embodiment 10. A modular port accessory device providing for one-time passage through the myocardium for removal of a mitral valve clip and subsequent delivery of a mitral valve implantation the port accessory device comprising, an adapter body, the adapter body comprising, an elongate body having a proximal end portion and a distal end portion, wherein the proximal end portion comprises an internal cavity and the distal end portion comprises an internal bore, wherein the internal cavity comprises a valve receiving space and a flush chamber connected to a flush port, wherein a distal end of the distal end portion comprises a distal tip, an internal bore extending the length between a distal opening and flush chamber interface, a valve positioned within the valve receiving space, a cap configured to mate with the adapter body to secure the valve within the valve receiving space, a flush tube selectively connected to the flush port, and a seal positioned on the distal tip, wherein the port accessory device is modular for configuration on a catheter assembly.

Embodiment 11. The port accessory device as recited in Embodiment 10, wherein a stop on the distal end portion defines the distal end of the distal end portion and the proximal end of the distal tip.

Embodiment 12. The port accessory device as recited in any of embodiments 10-11, wherein the distal tip includes an annular edge distal to the stop, the edge and stop together forming a sealing groove.

Embodiment 13. The port accessory device as recited in any of embodiments 10-12, wherein the seal is disposed within the sealing groove. The cross section of the seal can be any shape: square, rectangle or circle.

Embodiment 14. The port accessory device as recited in any of embodiments 10-13, wherein the distal end portion includes a connecting portion configured to mate with the catheter assembly.

Embodiment 15. The port accessory device as recited in any of embodiments 10-14, wherein the connecting portion comprises a keyed fit structure configured to interface with the catheter assembly.

Embodiment 16. The port accessory device as recited in any of embodiments 10-15, wherein the connecting portion comprises a threaded section.

Embodiment 17. The port accessory device as recited in any of embodiments 10-16, wherein the cap has an opening configured to align with the internal bore of the adapter body.

Embodiment 18. A method for removal of a mitral valve clip using a modular adapter and subsequent delivery of a mitral valve implantation in a mitral valve replacement procedure, the method comprising, gaining access to a left atrium of a heart using standard techniques to position a guidewire in the left atrium, connecting the modular adapter to a catheter assembly, removing the air from the delivery sheath and from the modular adapter using the flush port, inserting a dilator device through a side port of the catheter assembly and inflating the dilator device to between 3 ATM to 8 ATM in a delivery sheath of the catheter assembly, advancing the catheter assembly and the modular adapter over the guidewire and then using the dilator device to dilate an access site and gain access to a left ventricle, advancing the catheter assembly and the modular adapter until a distal end portion of the delivery sheath passes a mitral valve, deflating and retracting the dilator device and guidewire from the side port and initiate a slow flush from the flush port and flush tube of the modular adapter, inserting a clip management tool through the port accessory device and through the catheter assembly, advancing the clip management tool until a tip of the clip management tool is in the left atrium, retracting the catheter assembly and modular adapter so that the distal end of the delivery sheath is in the left ventricle while the clip management tool remains stationary, capturing the mitral valve clip and cutting the clip from the mitral valve with the clip management tool, advancing the catheter assembly and modular adapter so that the distal end of the delivery sheath is in the left atrium while the clip management tool remains stationary, retracting the clip management tool while flushing the port accessory device and the catheter assembly by flushing fluid through the flush tube and flush port into the modular adapter, inserting a transition balloon device through the catheter assembly side port after the clip management tool is removed from the catheter assembly and the modular adapter, inflating the transition balloon device to between 3 ATM to 8 ATM in the delivery sheath, removing the modular adapter from the catheter assembly, connecting a valve holding tube and handle assembly loaded with the mitral valve implantation to the catheter assembly, forming a delivery system, removing air from the delivery system, deflating and removing the transition balloon device from the side port, completing the mitral valve implantation delivery procedure per instructions for use.

Embodiment 19. The method of as recited in embodiment 18, wherein the modular adapter is the modular adapter of any of embodiments 1-9.

Embodiment 20. The method as recited in any of embodiments 18-19, wherein the port accessory device is the device as recited in any of embodiments 10-17.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for removal of a fixation device from a valve of a heart and subsequent delivery of a valve implant through a single catheter access, comprising:
connecting a modular adapter to a connector of a catheter assembly, the connector configured to alternatively receive the modular adapter and a valve delivery device configured to carry the valve implant;
advancing the catheter assembly into the heart;
inserting a removal tool through the modular adapter and into the heart;
removing the fixation device from the valve using the removal tool;
disconnecting the modular adapter from the connector while the catheter assembly remains positioned within the heart;
connecting the valve delivery device to the connector; and
delivering the valve implant through the catheter assembly.

2. The method of claim 1, wherein the modular adapter includes a hemostatic valve disposed therein, and wherein disconnecting the modular adapter from the connector removes the hemostatic valve from the catheter assembly.

3. The method of claim 2, wherein the inserting step includes passing the removal tool through the hemostatic valve.

4. The method of claim 2, further comprising, prior to the disconnecting step, inflating a transition balloon within a delivery sheath of the catheter assembly to maintain hemostasis subsequent to disconnection of the modular adapter.

5. The method of claim 4, further comprising inserting the transition balloon through a side port of a hub of the catheter assembly, the hub being disposed distal to the connector, and the delivery sheath extending distally from the hub.

6. The method of claim 4, further comprising deflating and removing the transition balloon after connecting the valve delivery device to the connector of the catheter assembly.

7. The method of claim 4, wherein the transition balloon is inflated to between 3 ATM and 8 ATM.

8. The method of claim 1, wherein the valve delivery device comprises a handle assembly and a valve holding tube, the valve holding tube being connectable to the connector of the catheter assembly.

9. The method of claim 1, wherein the modular adapter includes a connecting portion having a threaded section and a key, and wherein the connecting step includes:
threadedly engaging the threaded section with the connector; and
aligning the key with a complementary receiving structure on a hub of the catheter assembly, the hub being disposed distal to the connector.

10. The method of claim 9, wherein the modular adapter further includes a distal tip extending distally from the connecting portion and a seal disposed on the distal tip, and wherein the connecting step further includes inserting the distal tip into the hub such that the seal is compressed within the hub to form a leak-proof seal between the modular adapter and the catheter assembly.

11. A method for removal of a fixation device from a valve of a heart and subsequent delivery of a valve implant through a single catheter access, comprising:
connecting a modular adapter to a connector of a catheter assembly, the modular adapter having a hemostatic valve disposed therein, and the connector being configured to alternatively receive the modular adapter and a valve delivery device configured to carry the valve implant;
advancing the catheter assembly into the heart;
inserting a removal tool through the hemostatic valve of the modular adapter and into the heart;
removing the fixation device from the valve using the removal tool;
disconnecting the modular adapter from the connector while the catheter assembly remains positioned within the heart, wherein the hemostatic valve is removed from the catheter assembly with the modular adapter;
connecting the valve delivery device to the connector; and
delivering the valve implant through the catheter assembly.

12. The method of claim 11, further comprising, prior to the disconnecting step, inflating a transition balloon within a delivery sheath of the catheter assembly to maintain hemostasis subsequent to disconnection of the modular adapter.

13. The method of claim 12, further comprising deflating and removing the transition balloon after connecting the valve delivery device.

14. The method of claim 11, wherein the modular adapter includes a connecting portion having a threaded section, and wherein the connecting step includes threadedly engaging the threaded section with the connector.

15. The method of claim 11, wherein the valve delivery device comprises a handle assembly and a valve holding tube, the valve holding tube being configured to connect to the connector of the catheter assembly.

16. The method of claim 11, wherein the hemostatic valve comprises a slit, and inserting the removal tool comprises passing the removal tool through the slit.

17. The method of claim 11, wherein the modular adapter includes a seal positioned on a distal tip of the modular adapter, the seal being configured to form a leak-proof seal with the catheter assembly when the modular adapter is connected to the connector.

18. A system for removal of a fixation device from a valve of a heart and subsequent delivery of a valve implant, comprising:
a catheter assembly comprising a delivery sheath and a connector disposed at a proximal end of the delivery sheath, the delivery sheath configured to be advanced into the heart;
a modular adapter comprising an adapter body having a connecting portion configured to selectively connect to and disconnect from the connector of the catheter assembly, the modular adapter configured to receive a removal tool therethrough for removal of the fixation device; and
a valve delivery device comprising a valve holding tube configured to carry the valve implant, the valve holding tube having a connecting portion configured to selectively connect to the connector of the catheter assembly;
wherein the connector is configured to alternatively receive the connecting portion of the modular adapter and the connecting portion of the valve holding tube.

19. The system of claim 18, wherein the modular adapter comprises a hemostatic valve disposed therein, and wherein upon disconnection of the modular adapter from the connector, the hemostatic valve is removed from the catheter assembly.

20. The system of claim 18, wherein the valve delivery device further comprises a handle assembly coupled to the valve holding tube, the valve holding tube being configured to hold the valve implant.
